(19) United States
(12) Patent Application Publication
(10) Pub. No.: US 2023/0010349 A1
(43) Pub. Date: Jan. 12, 2023

(54) Novel Multipurpose Fast Dehumidification Device

(71) Applicant: KONTROL GROUP LIMITED, Shrewsbury (GB)

(72) Inventors: Keith William Alfred Cochrane, Shrewsbury (GB); Kenneth William Bates, Shrewsbury (GB); Stephen Anthony Woodland, Shrewsbury (GB)

(21) Appl. No.: 17/370,059

(22) Filed: Jul. 8, 2021

(51) Int. Cl. B01D 53/26; B01D 53/04; A01M 29/12; A61L 9/16

(52) U.S. Cl. CPC B01D 53/261; B01D 53/0454; B01D 53/0446; A01M 29/12; A61L 9/16; B01D 2257/80; A01M 2200/012; A61L 2209/11

(57) ABSTRACT

The present invention discloses a novel multipurpose fast dehumidification device, in which materials with different functions can be placed simultaneously or separately, such as moisture absorption materials, aromatic materials, mosquito repelling (insect repelling) materials or deodorization materials. The present invention comprises a base, a placement frame and a circulation mechanism One end of the placement frame is movably connected with the base, and the other end of the placement frame is movably connected with the circulation mechanism; and the moisture absorption materials are placed in the placement frame. After the device is started, air in an environment is sucked by the circulation mechanism, then reaches the placement frame, and then is discharged from the placement frame after being dehumidified. The present invention has the beneficial effects that the structure is simple, and the use is convenient; the complicated structure of a traditional dehumidifier is abandoned, and the cost of a product is reduced to a maximum degree; at the same time, a single dehumidification function is expanded, which can play a role in different use scenes. That is, besides the dehumidification function, functions of atmosphere creation, mosquito repelling (insect repelling), deodorization and the like can also be realized.

### TECHNICAL FIELD

The present invention belongs to the technical field of a dehumidification device in daily chemical products, and more particularly relates to a novel multipurpose fast dehumidification device.

### BACKGROUND ART

A dehumidifier is one of the common products at home, especially in the south with high humidity. Humid air in “back to the south weather” every year will cause water droplets to appear on floor tiles and wall surfaces, and clothes are difficult to dry in this environment. Therefore, the occurrence of the dehumidifier greatly solves the above problems, but existing dehumidifiers generally include the following two types:

1. A condensation dehumidifier, characterized in that humid air is sucked into the dehumidifier; then, moisture in the air is condensed and collected into a water tank through an evaporator and a condenser; and next, dry hot air is discharged to achieve a goal of dehumidification. The condensation dehumidifier has the defects of relatively big noise and generally working in a range of 5° C. to 35° C. At present, most dehumidifiers on the market are condensation dehumidifiers.

2. A rotary dehumidifier, characterized in that its principle is opposite to that of the condensation dehumidifier. The rotary dehumidifier heats up and condenses the air, absorbs moisture in the air through a dehumidification rotary wheel, then takes away the moisture from the rotary wheel by using the heated dry air, and next condenses this part of high-temperature wet air to achieve the goal of dehumidification. The rotary dehumidifier has the defects of relatively weak dehumidification capacity, high energy consumption and high price.

Although the above dehumidifiers have their own advantages, wide-range popularization is restricted by the high price of the product and the limitation of the use scene. Therefore, whether a dehumidifier with low cost and high practicability can be developed or not becomes the focus of the present invention.

### SUMMARY OF THE INVENTION

In view of the defects in the prior art, the present invention aims to provide a novel multipurpose fast dehumidification device. Moisture absorption materials can be directly placed in the device; and then, humid air in an environment is continuously sucked into the device through an electronic fan, so that a dehumidification effect of the moisture absorption materials is accelerated. At the same time, aromatic materials, mosquito repelling (insect repelling) materials and deodorization materials can also be placed in the device. When dehumidification is not required or while dehumidifying, the present device is used to create an environment atmosphere, repel mosquitoes (insects) and remove odor.

In order to achieve the above objective, the present invention adopts the following technical solution:

A novel multipurpose fast dehumidification device is provided with different types of moisture absorption materials, aromatic materials, mosquito repelling (insect repelling) materials or deodorization materials, and includes a base, a placement frame and a circulation mechanism One end of the placement frame is movably connected with the base, and the other end of the placement frame is movably connected with the circulation mechanism The moisture absorption materials are placed in the placement frame. After the device is started, air in an environment is sucked by the circulation mechanism, then reaches the placement frame, and then is discharged from the placement frame after being dehumidified.

It should be noted that a bottom of the placement frame is provided with a lug boss extending outward. When the placement frame is movably connected with the base, the lug boss is inserted into the base, wherein an outer surface of a peripheral edge of the lug boss is tightly connected with a top inner surface of the base.

As a preferable technical solution, a peripheral edge of an outer surface of the lug boss is provided with first flanges; and the top inner surface of the base is provided with first grooves corresponding to positions of the first flanges. When the placement frame is movably connected with the base, the first flanges are buckled into the first grooves.

It should be further noted that the circulation mechanism includes a casing and a control unit disposed in the casing. An inner surface of a top peripheral edge of the placement frame is provided with second flanges, and an outer surface of a bottom peripheral edge of the casing is provided with second grooves corresponding to positions of the second flanges. When the placement frame is movably connected with the casing, the inner surface of the top peripheral edge of the placement frame is tightly connected with the outer surface of the bottom peripheral edge of the casing, and the second flanges are buckled into the second grooves.

It should be pointed out that the arrangement positions of the first flanges and the first grooves of the present invention can be interchanged. That is, in one solution, the first flanges may be disposed on the base, and the first grooves may be formed in the lug boss; and similarly, the second flanges and the second grooves of the present invention may also realize the above deformation.

It should be further pointed out that the first flanges, the first grooves, the second flanges and the second grooves have the same technical conceptions as the male and female interlocking mechanism in the prior art.

It should be further noted that the control unit includes an electronic fan, and a control circuit configured to control a rotating speed and an operating time of the electronic fan.

It should be pointed out that the control unit is identical to an existing control unit, and may be provided with an indicating lamp to indicate a use state, and also has mode selection. Further, the mode selection in a preferable solution may be different modes corresponding to different rotating speeds of the electronic fan or controlled operation in a specific operation time.

It should be pointed out that if the present invention is further optimized, a humidity sensor or a WIFI module may be additionally disposed to realize environment humidity monitoring, so that intelligent regulation and control is realized.

It should be further pointed out that a power supply in the control unit of the present invention may be realized by using a lithium battery.

As another preferable technical solution, the present invention further includes a function mechanism. The function mechanism includes a fixing plate and a placement box. One side of the fixing plate is fixedly connected with a bottom of the electronic fan, and the placement box is fixedly connected with the other side of the fixing plate. A surface of the fixing plate is provided with a plurality of through holes.

It should be pointed out that in order to conveniently place the aromatic materials, the mosquito repelling (insect repelling) materials or the deodorization materials into the placement box, the fixing plate and the placement box of the present invention may be of an integrally formed structure. Through a folding mark reserved between an edge of the fixing plate and an edge of the placement box, the fixing plate and the placement box may be relatively turned over to form an open/closed integral structure. Certainly, in order to ensure avoiding a falling phenomenon after the fixing plate and the placement box are closed, buckling members may be disposed in the placement box. When the fixing plate and the placement box are closed, the buckling members are buckled into through holes formed in the fixing plate.

As a preferable technical solution, in order to realize a more compact structure, the control circuit of the present invention may be connected with the fixing plate. Specifically, the bottom of the fixing plate may be provided with a pair of slide grooves, and the control circuit is slidingly connected into the slide grooves, so that not only the structure is further optimized, but also fixation by screws is reduced, which effectively reduces the production cost. At the same time, a battery electrically connected with the electronic fan may also be disposed in the placement box, so that the structure of the present invention is further compact.

Similarly, as a deformation of the structure of the fixing plate and the placement box, the placement box may be provided with an open-close type cover plate. The cover plate and the placement box may be made by an integral forming process. Through the folding mark reserved between an edge of the cover body and an edge of the placement box, the cover body may be turned over relative to the placement box.

Further, in order that the aromatic materials, the mosquito repelling (insect repelling) materials or the deodorization materials may be fast diffused, a surface of the placement box is also provided with a plurality of through holes.

It should be noted that a peripheral edge of a surface of the placement frame is provided with a plurality of slots and/or open holes for discharging air, and a surface of the lug boss is provided with a plurality of slots and/or open holes communicating with the base.

It should be pointed out that the base is provided with a chamber for storing water, and a surface of the base is provided with scale lines for observing a water level.

As a preferable technical solution, the base in the present invention may be made of a transparent material, so that the water level in the chamber for storing water may be conveniently observed; or when the base is made of a non-transparent material, a water level view window may be disposed at one side of the base, which can also play a role in observing the water level in the chamber for storing water.

As another preferable technical solution, a top peripheral edge of the base in the present invention may also be provided with a drain opening for pouring water. When water collected in the base has exceeded a warning line of the scale, all water in the base may be poured out through the drain opening.

It should be pointed out that the moisture absorption materials used in the present invention may be materials with dehumidification capabilities such as dehumidification packets, dehumidification bags, dehumidification cakes or dehumidification beads. At the same time, if the dehumidification effect needs to be enhanced, 2 or more moisture absorption materials may be simultaneously placed into the placement frame of the present invention.

As another preferable technical solution, when a plurality of moisture absorption materials are placed into the placement frame, in order to ensure the previous moisture absorption capability thereof not to be affected, a separation plate vertical to the lug boss may be disposed in the placement frame, so that the plurality of moisture absorption materials may be separated from each other. At the same time, a surface of the separation plate may also be provided with slots and/or open holes.

In another solution, if the moisture absorption material is a dehumidification cake, a dismountable fixing post may be disposed in the lug boss, and then, the dehumidification cake is inserted into the fixing post to be fixed.

In another solution, if the moisture absorption material is a dehumidification cake, several supporting posts may be disposed in the lug boss, and then the dehumidification cake is placed on the several supporting posts such that a certain distance is formed between the bottom of the dehumidification cake and the lug boss, which may increase the contact area of the dehumidification cake with humid air, and at the same time the air flow efficiency of the entire dehumidification cake is improved.

Certainly, in order to meet the dehumidification requirements of different customers, the separation plate should be movably connected with the lug boss, so that mounting and dismounting are convenient.

As a preferable technical solution, when the present invention needs to achieve an effect of environment atmosphere creation, mosquito repelling (insect repelling) or deodorization, the aromatic materials, the mosquito repelling (insect repelling) materials or the deodorization materials may be placed into the placement box. Since the placement box is close to the electronic fan, aromatic odor or mosquito repelling (insect repelling) gas or deodorization gas can be emitted to an ambient environment at a highest speed.

The aromatic materials referred to in the present invention include aromatic packets, aromatic bags or aromatic pendants and the like. The mosquito repelling (insect repelling) materials referred to in the present invention include mosquito repelling (insect repelling) tablets, mosquito repelling (insect repelling) packets, mosquito repelling (insect repelling) bags or mosquito repelling (insect repelling) pendants. The deodorization materials referred to in the present invention include deodorization packets, deodorization bags or deodorization pendants and the like.

Certainly, if an environment space is relatively large, or the aromatic, mosquito repelling (insect repelling) or deodorization effects need to be fast achieved, multiple materials may be placed into it. Since the packaging specifications of existing materials are different, the materials are pendants, which may be hung on the plurality of through holes formed in the fixing plate or hung on the placement box.

As a preferable technical solution, a bottom of the base of the present invention may also be provided with support feet.

The present invention has the beneficial effects that the structure is simple, and the use is convenient; the complicated stricture of a traditional dehumidifier is abandoned, and the cost of a product is reduced to a maximum degree; at the same time, a single dehumidification function is expanded, which can play a role in different use scenes. That is, besides the dehumidification function, functions of atmosphere creation, mosquito repelling (insect repelling), deodorization and the like can also be realized.

### BRIEF DESCRIPTION OF THE DRAWINGS

FIG. 1 is a schematic structure diagram of the present invention;

FIG. 2 is a schematic structure diagram of a placement frame in FIG. 1;

FIG. 3 is a schematic structure diagram of the placement frame in FIG. 2 in another angle;

FIG. 4 is a schematic structure diagram of a base in FIG. 1;

FIG. 5 is a schematic structure diagram of a circulation mechanism in FIG. 1;

FIG. 6 is a schematic diagram of mechanisms of an electronic fan and a control unit in FIG. 5;

FIG. 7 is a schematic structure diagram of function mechanisms in FIG. 6;

FIG. 8 is a schematic diagram of a preferred structure mode of a fixing plate and an placement box in FIG. 7; and

FIG. 9 is a schematic structure diagram of a closed state of the fixing plate and the placement box in FIG. 8.

### DETAILED DESCRIPTION OF THE INVENTION

Hereinafter, the present invention will be further described with reference to the accompanying drawings. It should be noted that the present embodiment provides a detailed implementation manner and a specific operation process on the basis of the present technical solution, but the protection scope of the present invention is not limited to the present embodiment.

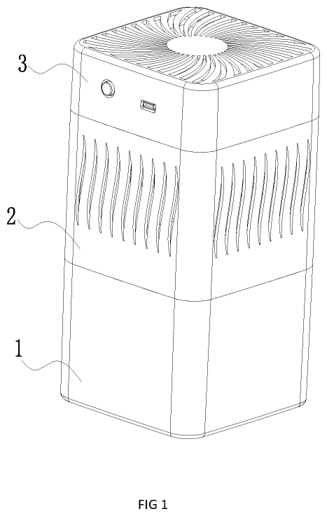

As shown in FIG. 1, the present invention provides a novel multipurpose fast dehumidification device, provided with different types of moisture absorption materials, aromatic materials, mosquito repelling (insect repelling) materials or deodorization materials. The device includes a base 1, a placement frame 2 and a circulation mechanism 3 One end of the placement frame 2 is movably connected with the base 1, and the other end of the placement frame is movably connected with the circulation mechanism 3. The moisture absorption materials are placed into the placement frame 2. After the device is started, air in an environment is sucked by the circulation mechanism 3, then reaches the placement frame 2, and then is discharged from the placement frame 2 after being dehumidified.

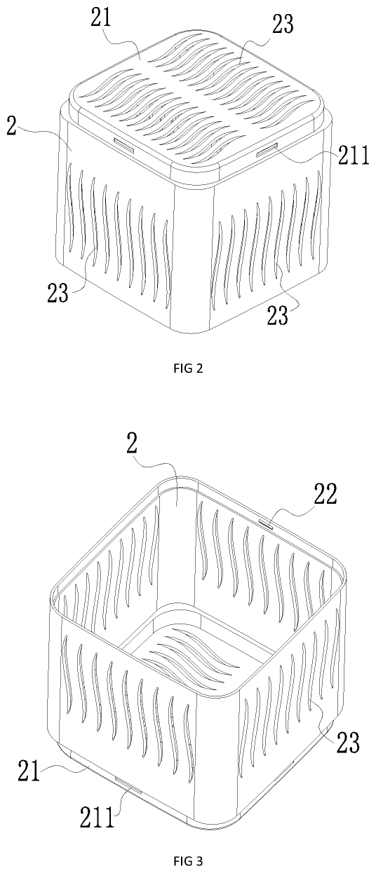

As shown in FIG. 2 and FIG. 3, a bottom of the placement frame 2 of the present invention is provided with a lug boss 21 extending outward. When the placement frame 2 is movably connected with the base, the lug boss 21 is inserted into the base, wherein an outer surface of a peripheral edge of the lug boss 21 is tightly connected with a top inner surface of the base.

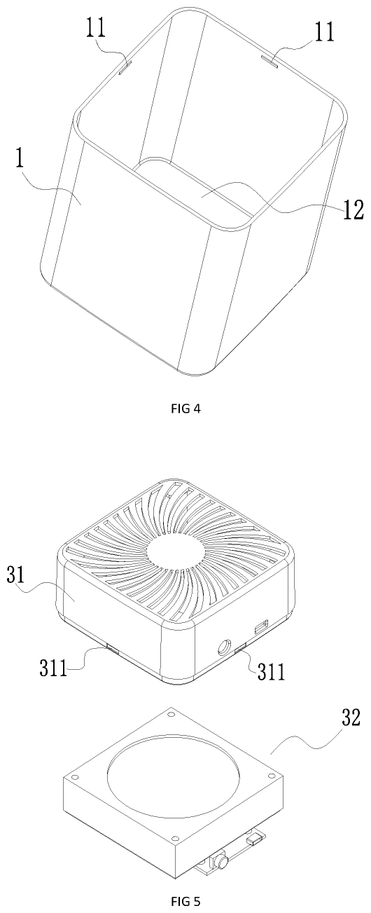

As shown in FIG. 2, FIG. 3 and FIG. 4, a peripheral edge of an outer surface of the lug boss 21 is provided with first flanges 211. The top inner surface of the base 1 is provided with first grooves 11 corresponding to positions of the first flanges 211. When the placement frame 2 is movably connected with the base 1, the first flanges 211 are buckled into the first grooves 11.

As shown in FIG. 3 and FIG. 5, the circulation mechanism 3 includes a casing 31 and a control unit 32 disposed in the casing 31. An inner surface of a top peripheral edge of the placement frame 2 is provided with second flanges 22. An outer surface of a bottom peripheral edge of the casing 31 is provided with second grooves 311 corresponding to positions of the second flanges 22. When the placement frame 2 is movably connected with the casing 31, the inner surface of the top peripheral edge of the placement frame 2 is tightly connected with the outer surface of the bottom peripheral edge of the casing 31, and the second flanges 22 are buckled and the second grooves 311.

It should be pointed out that the arrangement positions of the first flanges and the first grooves of the present invention can be interchanged. That is, in one solution, the first flanges may be disposed on the base, and the first grooves may be formed in the lug boss; and similarly, the second flanges and the second grooves of the present invention may also realize the above deformation.

It should be further pointed out that the first flanges, the first grooves, the second flanges and the second grooves have the same technical conceptions as the male and female interlocking mechanism in the prior art.

Besides the above connecting mode of the base, the placement frame and the circulation mechanism, the present invention further provides another preferable technical solution, and first magnetic members in corresponding positions are respectively disposed on the peripheral edge of the outer surface of the lug boss and the top inner surface of the base. When the placement frame is movably connected with the base, the first magnetic members are mutually and magnetically connected.

It should be further noted that the circulation mechanism includes a casing and a control unit disposed in the casing. An inner surface of a top peripheral edge of the placement frame and an outer surface of a bottom peripheral edge of the casing are respectively provided with second magnetic members in corresponding positions. When the placement frame is movably connected with the casing, the inner surface of the top peripheral edge of the placement frame is tightly connected with the outer surface of the bottom peripheral edge of the casing, and the second magnetic members are mutually and magnetically connected.

As a preferable technical solution, a bottom of the base of the present invention may also be provided with support feet.

As shown in FIG. 6, the control unit 32 includes an electronic fan 321, and a control circuit 322 configured to control a rotating speed and an operating time of the electronic fan 321.

It should be pointed out that the control unit in the present invention is identical to an existing control unit, and may be provided with an indicating lamp to indicate a use state, and also has mode selection. Further, the mode selection in a preferable solution may be different modes corresponding to different rotating speeds of the electronic fan or controlled operation in a specific operation time.

It should be pointed out that if the present invention is further optimized, a humidity sensor or a WIFI module may be additionally disposed to realize environment humidity monitoring, so that intelligent regulation and control is realized.

It should be further pointed out that a power supply in the control unit of the present invention may be realized by using a lithium battery.

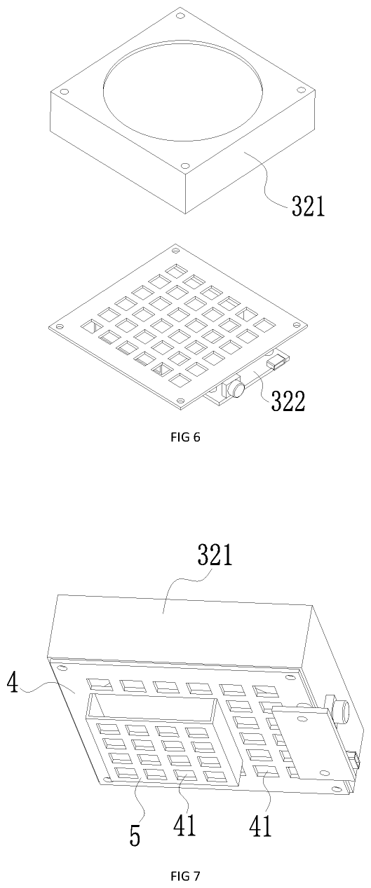

As shown in FIG. 7, as another preferable technical solution, the present invention further includes a function mechanism. The function mechanism includes a fixing plate 4 and a placement box 5. One side of the fixing plate 4 is fixedly connected with to bottom of the electronic fan 321, and the placement box 5 is fixedly connected with the other side of the fixing plate 4. A surface of the fixing plate 4 is provided with a plurality of through holes 41.

As shown in FIG. 8, in order to conveniently place the aromatic materials, the mosquito repelling (insect repelling) materials or the deodorization materials into the placement box 5, the fixing plate 4 and the placement box 5 of the present invention may be of an integrally formed structure. Through a folding mark reserved between an edge of the fixing plate 4 and an edge of the placement box 5, the fixing plate and the placement box may be relatively turned over to form an open/closed integral structure. Certainly, in order to ensure avoiding a falling phenomenon after the fixing plate 4 and the placement box 5 are closed, buckling members 51 may be disposed in the placement box 5. When the fixing plate 4 and the placement box 5 are closed, the buckling members 51 are buckled into through holes 41 formed in the fixing plate 4.

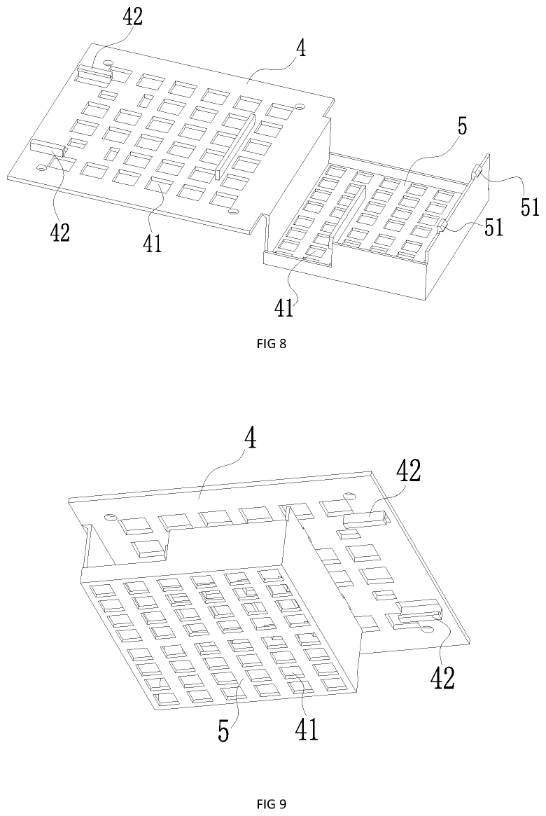

As shown in FIG. 8 and FIG. 9, as a preferable technical solution, in order to realize a more compact structure, the control circuit of the present invention may be connected with the fixing plate 4. Specifically, the bottom of the fixing plate 4 may be provided with a pair of slide grooves 42, and the control circuit is slidingly connected into the slide grooves 42, so that not only the structure is further optimized, but also fixation by screws is reduced, which effectively reduces the production cost. At the same time, a battery electrically connected with the electronic fan may also be disposed in the placement box, so that the structure of the present invention is further compact.

Similarly, as a deformation of the structure of the fixing plate and the placement box, the placement box may be provided with an open-close type cover plate. The cover plate and the placement box may be made by an integral forming process. Through the folding mark reserved between an edge of the cover body and an edge of the placement box, the cover body may be turned over relative to the placement box.

Further, as shown in FIG. 8 and FIG. 9, in order that the aromatic materials, the mosquito repelling (insect repelling) materials or the deodorization materials may be fast diffused, a surface of the placement box 5 is also provided with a plurality of through holes 41.

As shown in FIG. 2 and FIG. 3, a peripheral edge of a surface of the placement frame 2 is provided with a plurality of slots 23 and/or open holes (not shown in the figures) for discharging air, and a surface of the lug boss is provided with a plurality of slots 23 and/or open holes (not shown in the figures) communicating with the base.

It should be pointed out that as shown in FIG. 4, the base 1 is provided with a chamber 12 for storing water, and a surface of the base is provided with scale lines for observing a water level.

As a preferable technical solution, the base in the present invention may be made of a transparent material, so that the water level in the chamber for storing water may be conveniently observed; or when the base is made of a non-transparent material, a water level view window may be disposed at one side of the base, which can also play a role in observing the water level in the chamber for storing water.

As another preferable technical solution, a top peripheral edge of the base in the present invention may also be provided with a drain opening for pouring water. When water collected in the base has exceeded a warning line of the scale, all water in the base may be poured out through the drain opening.

It should be pointed out that the moisture absorption materials used in the present invention may be materials with dehumidification capabilities such as dehumidification packets, dehumidification bags, dehumidification cakes or dehumidification beads. At the same time, if the dehumidification effect needs to be enhanced, 2 or more moisture absorption materials may be simultaneously placed into the placement frame of the present invention.

As another preferable technical solution, when a plurality of moisture absorption materials are placed into the placement frame, in order to ensure the previous moisture absorption capability thereof not to be affected, a separation plate vertical to the lug boss may be disposed in the placement frame, so that the plurality of moisture absorption materials may be separated from each other. At the same time, a surface of the separation plate may also be provided with slots and/or open holes.

In another solution, if the moisture absorption material is a dehumidification cake, a dismountable fixing post may be disposed in the lug boss, and then, the dehumidification cake is inserted into the fixing post to be fixed.

In another solution, if the moisture absorption material is a dehumidification cake, several supporting posts may be disposed in the lug boss, and then the dehumidification cake is placed on the several supporting posts such that a certain distance is formed between the bottom of the dehumidification cake and the lug boss, which may increase the contact area of the dehumidification cake with humid air, and at the same time the air flow efficiency of the entire dehumidification cake is improved.

Certainly, in order to meet the dehumidification requirements of different customers, the separation plate should be movably connected with the lug boss, so that mounting and dismounting are convenient.

As a preferable technical solution, when the present invention needs to achieve an effect of environment atmosphere creation, mosquito repelling (insect repelling) or deodorization, the aromatic materials, the mosquito repelling (insect repelling) materials or the deodorization materials may be placed into the placement box. Since the placement box is close to the electronic fan, aromatic odor or mosquito repelling (insect repelling) gas or deodorization gas can be emitted to an ambient environment at a highest speed.

The aromatic materials referred to in the present invention include aromatic packets, aromatic bags or aromatic pendants and the like. The mosquito repelling (insect repelling) materials referred to in the present invention include mosquito repelling (insect repelling) tablets, mosquito repelling (insect repelling) packets, mosquito repelling (insect repelling) bags or mosquito repelling (insect repelling) pendants. The deodorization materials referred to in the present invention include deodorization packets, deodorization bags or deodorization pendants and the like.

Certainly, if an environment space is relatively large, or the aromatic, mosquito repelling (insect repelling) or deodorization effects need to be fast achieved, multiple materials may be placed into it. Since the packaging specifications of existing materials are different, the materials are pendants, which may be hung on the plurality of through holes formed in the fixing plate or hung on the placement box.

Embodiment

Dehumidification Mode:

In use, a customer may place the moisture absorption materials according to practical requirements. The moisture absorption materials are directly placed into the placement frame; one end of the placement frame is inserted into the base, and then, the circulation mechanism is inserted into the other end of the placement frame, so that installation can be completed. When the power supply is turned on, the electronic fan continuously sucks humid air in the environment from the top of the casing of the circulation mechanism, and the humid air will be conveyed into the placement frame and be in contact with the moisture absorption materials therein. The dehumidification circulation is completed after the air dehumidified by the moisture absorption materials is discharged from the slots or open holes in the peripheral edge of the placement frame. Water after the moisture absorption by the moisture absorption materials is dripped into the chamber for storing water in the base to be stored. The customer can judge whether the chamber is fully filled with water or not according to a warning water line on the base.

Aromatic mode:

When an environment atmosphere needs to be created, 1 or more aromatic materials are placed into the placement box, and if the aromatic materials are in a pendant type, the aromatic materials may be hung on the fixing plate. When the power supply is turned on, the electronic fan continuously sucks air from the top of the casing of the circulation mechanism, and fast blows the air to the aromatic materials. After passing through the aromatic materials, the air carries aromatic agent molecules and is discharged out from the slots or open holes of the peripheral edge of the placement frame, so that the environment atmosphere can be created through the present invention.

Mosquito repelling (insect repelling) mode or deodorization mode

This mode is identical to the above dehumidification or aromatic mode. Volatile mosquito repelling (insect repelling) materials or deodorization materials may be placed into the placement box, and the mosquito repelling (insect repelling) mode or deodorization mode may be started according to the above working mode.

It should be pointed out that the aromatic mode, mosquito repelling (insect repelling) mode or deodorization mode may be performed with the dehumidification mode at the same time or performed separately.

Various corresponding changes may be made by those skilled in the art according to the above technical solutions and concepts, and all such changes shall be included within the protection scope of the appended claims of the present invention.

1. A novel multipurpose fast dehumidification device, provided with different types of moisture absorption materials, aromatic materials, mosquito repelling (insect repelling) materials or deodorization materials, wherein the device comprises a base, a placement frame and a circulation mechanism; one end of the placement frame is movably connected with the base, and the other end of the placement frame is movably connected with the circulation mechanism; the moisture absorption materials are placed into the placement frame; after the device is started, air in an environment is sucked by the circulation mechanism, then reaches the placement frame, and then is discharged from the placement frame after being dehumidified.

2. The novel multipurpose fast dehumidification device according to claim 1, wherein a bottom of the placement frame is provided with a lug boss extending outward; and when the placement frame is movably connected with the base, the lug boss is inserted into the base, wherein an outer surface of a peripheral edge of the lug boss is tightly connected with a top inner surface of the base.

3. The novel multipurpose fast dehumidification device according to claim 2, wherein a peripheral edge of an outer surface of the lug boss is provided with first flanges; the top inner surface of the base is provided with first grooves corresponding to positions of the first flanges;
and when the placement frame is movably connected with the base, the first flanges are buckled into the first grooves.

4. The novel multipurpose fast dehumidification device according to claim 1, wherein the circulation mechanism comprises a casing and a control unit disposed in the casing; an inner surface of a top peripheral edge of the placement frame is provided with second flanges, and an outer surface of a bottom peripheral edge of the casing is provided with second grooves corresponding to positions of the second flanges; and when the placement frame is movably connected with the casing, the inner surface of the top peripheral edge of the placement frame is tightly connected with the outer surface of the bottom peripheral edge of the casing, and the second flanges are buckled into the second grooves.

5. The novel multipurpose fast dehumidification device according to claim 4, wherein the control unit comprises an electronic fan, and a control circuit configured to control a rotating speed and an operating time of the electronic fan.

6. The novel multipurpose fast dehumidification device according to claim 5, further comprising a function mechanism, wherein the function mechanism comprises a fixing plate and a placement box; one side of the fixing plate is fixedly connected with a bottom of the electronic fan, and the placement box is fixedly connected with the other side of the fixing plate; a surface of the fixing plate is provided with a plurality of through holes; the aromatic materials, the mosquito repelling (insect repelling) materials or the deodorization materials are placed into the placement box; and the placement box is provided with an open-close type cover plate.

7. The novel multipurpose fast dehumidification device according to claim 2, wherein a peripheral edge of a surface of the placement frame is provided with a plurality of slots and/or open holes for discharging air; and a surface of the lug boss is provided with a plurality of slots and/or open holes communicating with the base.

8. The novel multipurpose fast dehumidification device according to claim 2 or 9, wherein the base is provided with a chamber for storing water; and a surface of the base is provided with scale lines for observing a water level.
